(19) United States
(12) Patent Application Publication
(10) Pub. No.: US 2009/0326867 A1
(43) Pub. Date: Dec. 31, 2009

(54) SIGNAL PROCESSING SYSTEMS AND METHODS FOR DETERMINING SLOPES OF ELECTRONIC SIGNALS

(75) Inventors: James Watson, Dunfermline (GB); Paul Stanley Addison, Edinburgh (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue Galway (IE)

(21) Appl. No.: 12/242,882

(22) Filed: Sep. 30, 2008

Related U.S. Application Data
(60) Provisional application No. 61/077,079, filed on Jun. 30, 2008.
(60) Provisional application No. 61/077,130, filed on Jun. 30, 2008.

(51) Int. Cl. G06F 17/18; A61B 5/145

(52) U.S. Cl. 702/180; 600/364

(57) ABSTRACT

The present disclosure relates to signal processing and, more particularly, relates to determining the slope of a signal. In embodiments, first slopes between a first point in the signal and other points in the signal may be determined. Second slopes between a second point in the signal and other points in the signal may also be determined. The first and second slopes may be used to generate a histogram, and a desired slope may be selected from the preferred value of the histogram. In an embodiment, a two-dimensional Lissajous figure may be selected from a three-dimensional Lissajous figure and a histogram of the slopes within the selected Lissajous figure may be created to determine the desired slope. The desired slope may have clinical relevance (e.g., it may be used to determine a patient's blood oxygen saturation level). The three-dimensional Lissajous figure may be derived at least in part from surface signals related to two transform-surfaces. Each transform-surface may be the result of performing a continuous wavelet transform on a signal. A confidence measure may be generated with respect to determining the desired slope.

### CROSS-REFERENCE TO RELATED APPLICATIONS

This application claims the benefit of U.S. Provisional Application No. 61/077,079, entitled, “Signal Processing Systems and Methods for Determining Slopes,” filed Jun. 30, 2008, and of U.S. Provisional Application No. 61/077,130, entitled “Systems and Methods of Signal Processing,” filed Jun. 30, 2008, which are hereby incorporated by reference herein in their entireties.

### SUMMARY

The present disclosure generally relates to signal processing systems and methods, and more particularly, to systems and methods for determining a slope of a signal. In an embodiment, a slope is determined by calculating, for each of a number of data points of the signal, the slope between the data point and a plurality of other data points of the signal. The calculated slopes are compared to determine a desired slope, such as the most common, or dominant, slope.

In some embodiments, each of the calculated slopes may be used to generate a histogram. The histogram may show a maximum value at the desired slope of the signal. The desired slope value may represent the dominant slope of the plotted signal, regardless of whether all of the data points in the signal were used to calculate the slope values. Other secondary slopes (e.g., slope values due to calculating the slope of the signal using outlying data points) may exist and may be represented on the histogram, for example, at values spaced apart from the maximum value and thus the secondary slopes may not affect the dominant slope value. In an embodiment, the secondary slope may be the desired slope and the dominant slope may be due to an erroneous (e.g., artifact) slope if, for example, an artifact dominates the signal.

In an embodiment, the desired slope may be determined using each data point of the plotted signal. Alternatively, in an embodiment, data points in close proximity to each other may be ignored in calculating the slopes to preemptively remove the effect of artifacts in the signal (e.g. noise) on the calculations. In another suitable embodiment, data points close in time or any other suitable unit of measure may be ignored in calculating the slopes. Therefore, flexibility may be allowed to choose whichever local maxima in the histogram are desired.

The foregoing slope determination method may be employed in any suitable context. In some embodiments, it is employed in a noise reduction algorithm. Data points related to ancillary slopes may be ignored. For purposes of clarity, and not by way of limitation, some embodiments disclosed herein may include a process for determining physiological parameters from wavelet-transformed signals, such as a photoplethysmograph (PPG) signal and, more particularly, is disclosed for determining oxygen saturation (SpO₂) from wavelet-transformed PPG signals. In such an approach, a three-dimensional Lissajous plot is derived at least in part from wavelet transforms of PPG signals (i.e., wavelet transforms of the red and infrared light signals). The Lissajous plot is probed to find a two-dimensional Lissajous plot with a maximum spread along its principal axis and a minimum spread along an axis orthogonal to the principal axis. A representative slope is calculated from the two-dimensional Lissajous plot using the method described herein. The slope is used to index into an SpO₂ lookup table or used in a calibration equation to determine the oxygen saturation level of a patient from whom the PPG signals were obtained.

In an embodiment, a method for determining a desired slope of a signal is provided. The method may include determining first slopes between a first point in the signal and at least two other points in the signal, determining second slopes between a second point in the signal and at least two other points in the signal, generating a histogram from the first slopes and the second slopes, and selecting the desired slope of the signal corresponding to a preferred value in the histogram.

In an embodiment, a system for determining a desired slope of a signal is provided. The system may include an input signal generator for generating a signal, a processor coupled to the input signal generator, and an output coupled to the processor. The output may be capable of displaying information based at least in part on the desired slope. The processor may be capable of determining first slopes between a first point in the signal and at least two other points in the signal, determining second slopes between a second point in the signal and at least two other points in the signal, generating a histogram from the first slopes and the second slopes, and selecting the desired slope of the signal corresponding to a preferred value in the histogram.

In an embodiment, a computer-readable medium for use in determining a desired slope of a signal is provided. The computer-readable medium may have computer program instructions recorded thereon for determining first slopes between a first point in the signal and at least two other points in the signal, determining second slopes between a second point in the signal and at least two other points in the signal, generating a histogram from the first slopes and the second slopes, and selecting the desired slope of the signal corresponding to a preferred value in the histogram.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with some embodiments;

FIG. 5(a) shows an illustrative schematic of a signal plotted in accordance with an embodiment;

FIG. 5(b) shows a histogram of the slopes of the signal plotted in FIG. 5(a) in accordance with an embodiment;

FIG. 6 is a flowchart of an illustrative process for determining a desired slope of a signal in accordance with an embodiment;

FIG. 7 shows a plot of two signals detected in accordance with an embodiment;

FIG. 8 shows a transform-surface of each of the detected signals in FIG. 7 in accordance with an embodiment;

FIG. 9 shows a three-dimensional Lissajous figure derived at least in part from the transform-surfaces of FIG. 8 in accordance with an embodiment;

FIG. 10 shows a Lissajous figure selected from the three-dimensional Lissajous figure of FIG. 9 in accordance with an embodiment;

FIG. 11 shows a histogram of the slopes of the selected Lissajous figure of FIG. 10 in accordance with an embodiment;

FIG. 12 is a flowchart of an illustrative process for determining a blood oxygen saturation of a patient in accordance with an embodiment;

FIG. 13 is a flowchart of an illustrative process for determining a blood oxygen saturation of a patient after a noise algorithm is applied to a two-dimensional Lissajous figure in accordance with an embodiment; and

FIG. 14 is a flowchart of an illustrative process for generating a confidence measure in a histogram in accordance with an embodiment.

### DETAILED DESCRIPTION

The present disclosure generally relates to signal processing and, more particularly, the present disclosure relates to determining the slope of a signal such as, for example, a Lissajous figure derived from two photoplethysmograph (PPG) signals.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log Iₒ−(sβₒ+(1−s) βᵣ)l (2)
2. (2) is then differentiated with respect to time

$$\frac{d log I}{d t} = - ( s {β}_{o} + ( 1 - s ) \cdot {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

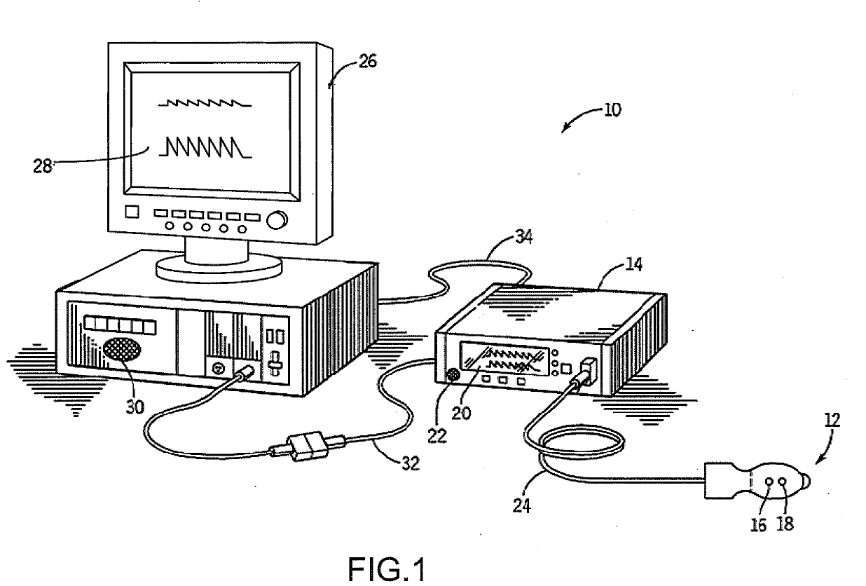

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to an embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

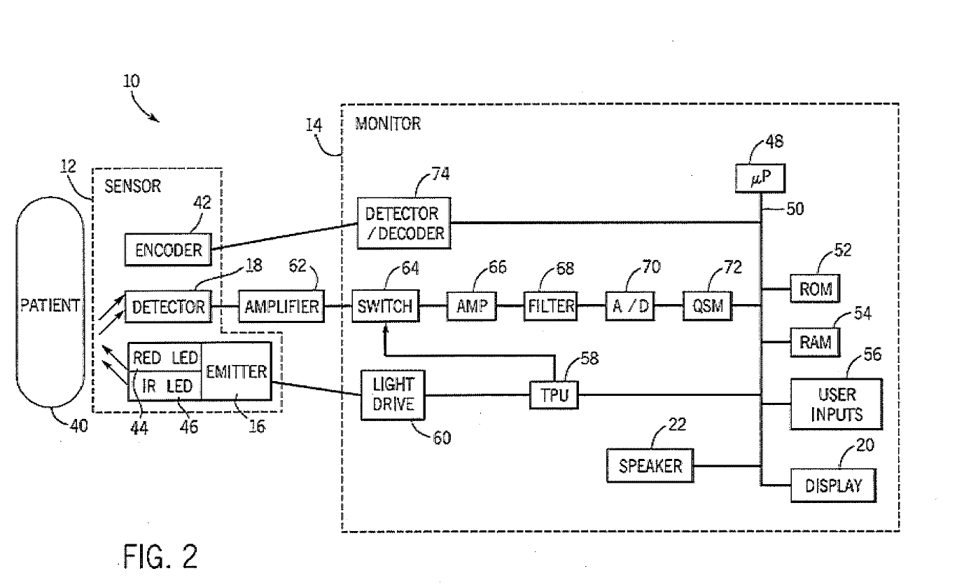

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be rescaled for useful purposes. One common rescaling is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

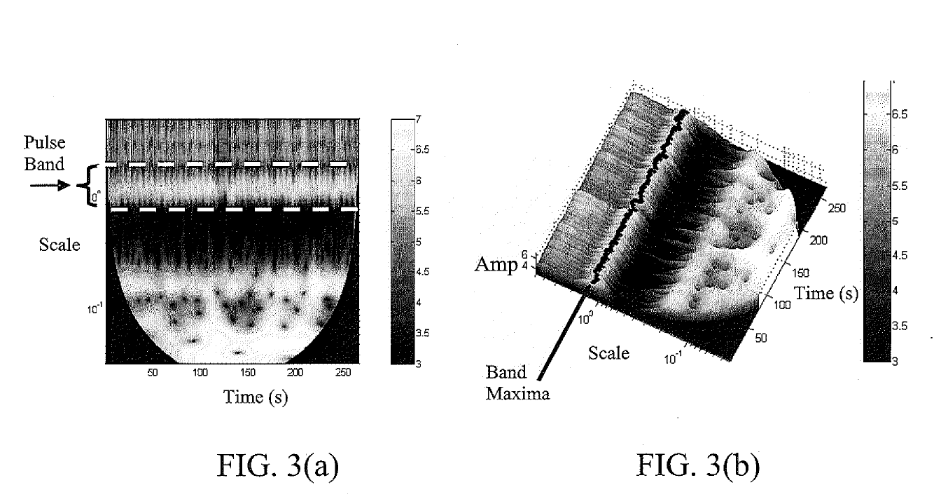

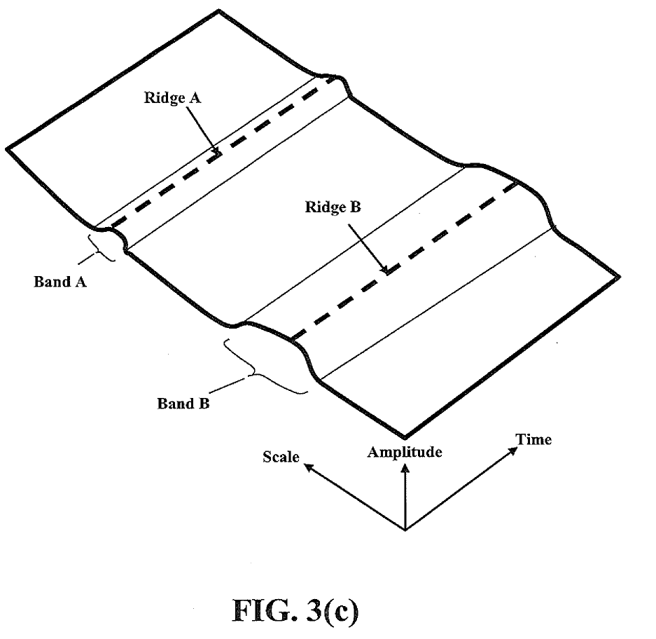

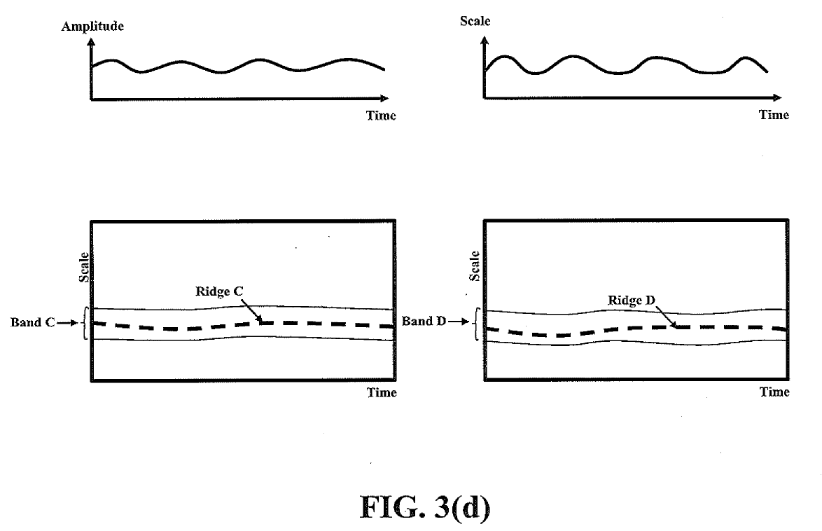

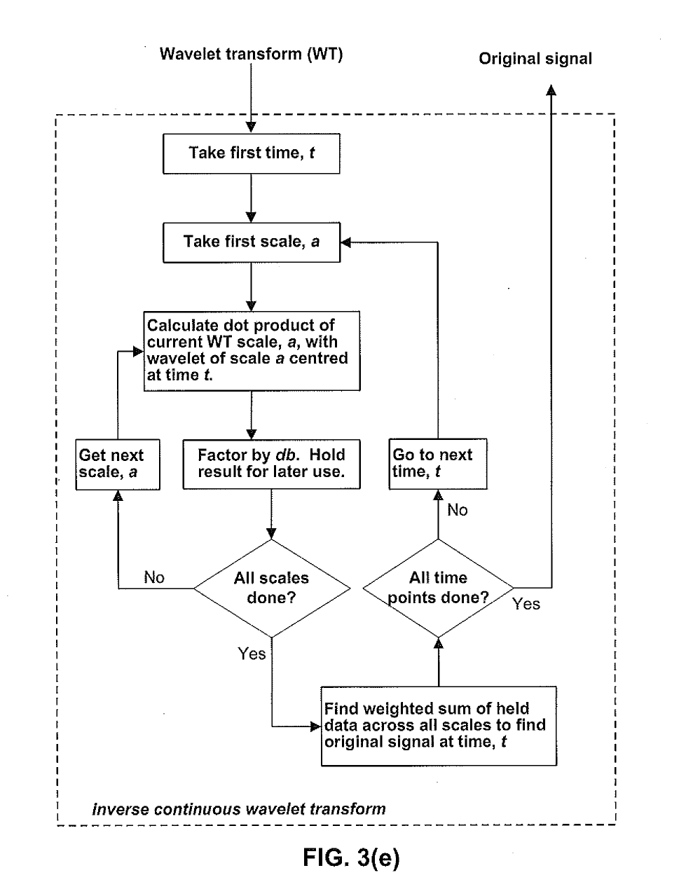

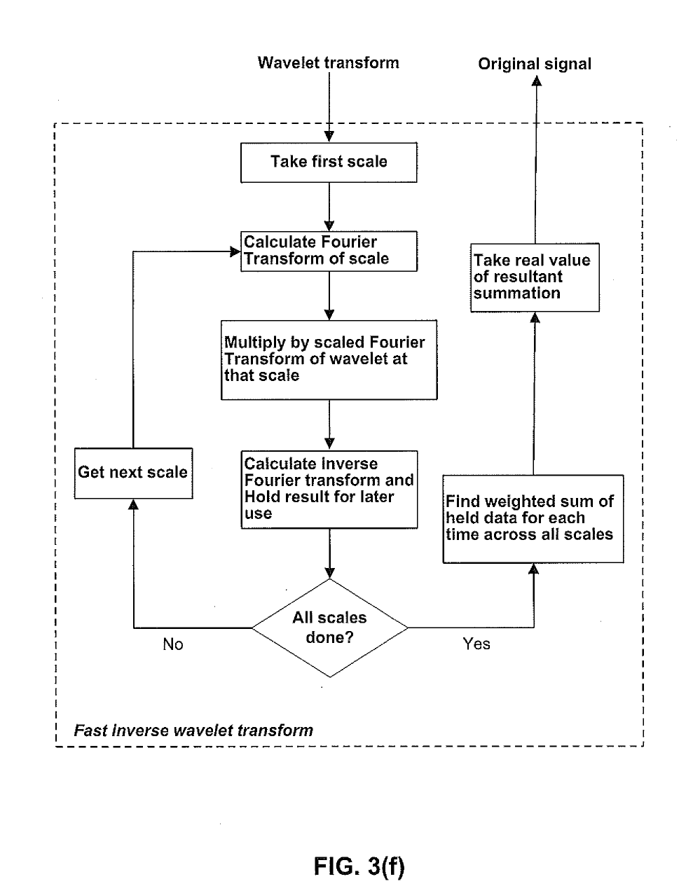

In embodiments, pertinent repeating features in a signal may give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

In embodiments, by mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some embodiments, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert }^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

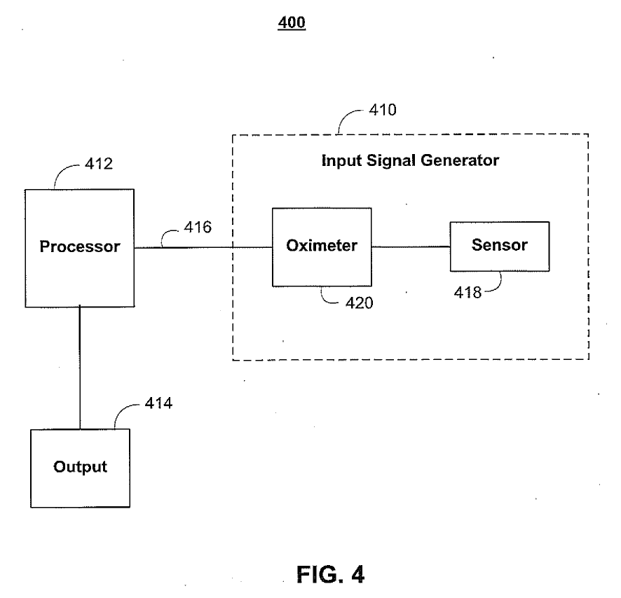

FIG. 4 is an illustrative continuous wavelet processing system 400 in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

In some embodiments, processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14, according to an embodiment.

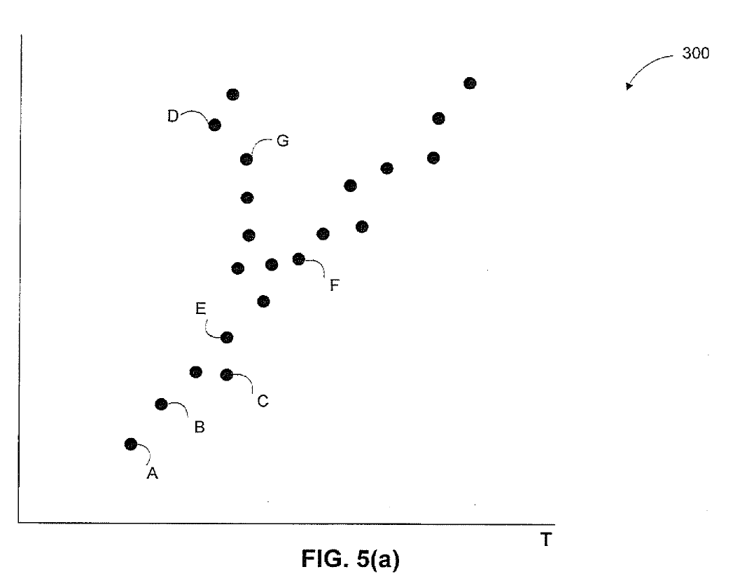

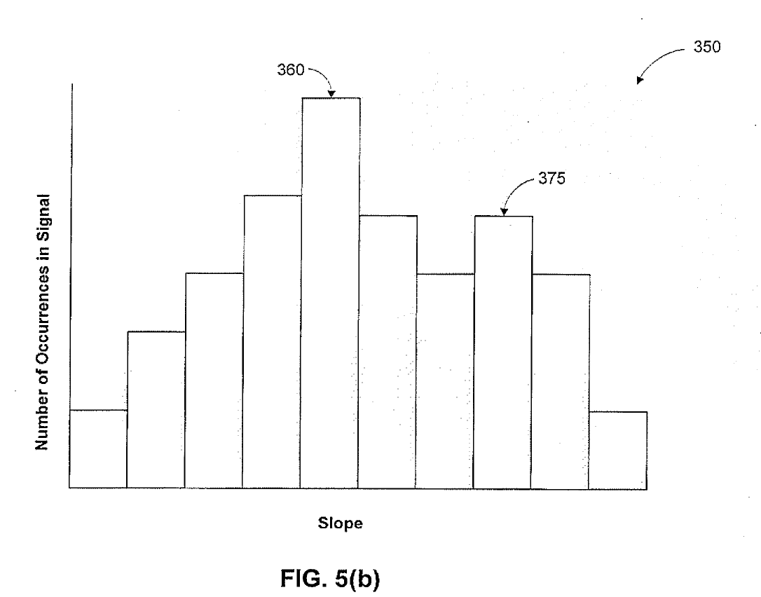

The slope determination process of the present disclosure will now be discussed in reference to FIGS. 5-14.

FIG. 5(a) shows an illustrative schematic of data points of a signal plotted in accordance with an embodiment of the disclosure. The signal may be any suitable signal, including a continuous signal, a discrete signal, or a signal formed from multiple data points. In an embodiment, a signal may include features of a scalogram or a Lissajous figure. In FIG. 5(a), plot 300 may include data points A, B, C, D, E, F, and G, and any other suitable number of data points within the signal. Plot 300 may include axes related to any suitable unit of measure, such as axes related to time, amplitude, scale, length, frequency, distance, or any other suitable unit of measure.

The slopes of the signal may be determined in any suitable manner. In some embodiments, a slope value may be determined between two points by taking the ratio of the rise from the first point to the second point to the run from the first point to the second point. The ratio may be taken with reference to any suitable axis (e.g., axis T, FIG. 5(a)). In some embodiments, a slope value may be determined by first determining the angle made between the same axis and a straight line connecting the first point and the second point. The determined angle may be thereafter converted to a slope value. If the slope value is determined from the second point to the first point, an angle may be similarly determined between axis T and the straight line connecting the second point and the first point. In some embodiments, determining angles and converting those angles into slope values may be more computationally efficient than determining the slope values because the angle values may span a narrower, linearly distributed range. For example, the angle values present between the points in the signal and the reference axis may span from −90° to 90° (passing through 0°), whereas the slope values may span from negative infinity to infinity (passing through zero). It is to be understood that the word “slope,” as used in this disclosure, may include any of the foregoing manners for determining a slope value.

In an embodiment, the slopes of the signal may be determined using each data point in plot 300. For example, a first slope of the signal may be determined between data point A and data point B, a second slope of the signal may be determined between data point A and data point C, and so on until each data point is used with each other data point to determine all of the slopes between the data points of the signal. Alternatively, in an embodiment, data points in close proximity to each other (e.g., data points C and E) may be ignored in calculating the slopes to preemptively remove the effect of artifacts in the signal (e.g., noise) on the calculations. In another suitable embodiment, data points close in time or any other suitable unit of measure may be ignored in calculating the slopes.

The slopes of the signal may also be determined between any data points of the signal, including, for example, between data points D and G, between data points D and other data points in the signal, and between data point G and other data points in the signal. Data points D and G may be “outlying” data points on plot 300, and may represent any suitable component within the signal, such as correlated noise. If outlying data points were used within an analysis of the slope of the plotted signal using a least squares technique, then the determined slope value may be skewed due to the presence of the outlying data points. A best fit line calculated by a least squares technique on plot 300 may be skewed (e.g., translated and rotated) from a proper best-fit line due to the presence of noise in the signal or other features of the signal as represented by the outlying data points.

In an embodiment, the presence of the outlying data points D and G may not affect the calculation of a desired slope (e.g., the dominant slope of the signal). As discussed above, slopes of the signal may be determined between each data point and each other data point of the signal. Each of the calculated slopes may be used to generate a histogram. The histogram may show a maximum value at the dominant slope of the signal. Other secondary slopes (e.g., slope values due to calculating the slope of the signal using outlying data points, such as data points D and G) may exist and may be represented on the histogram, for example, at values spaced apart from the maximum value and thus the secondary slopes may not affect the dominant slope value.

FIG. 5(b) shows a histogram of the slopes of the signal plotted in FIG. 5(a) in accordance with an embodiment of the disclosure. Histogram 350 may include any suitable axes representing any suitable units of measure. In an embodiment, histogram 350 may graph the number of occurrences of each calculated slope value or each calculated range of slope values between each data point and each other data point of plot 300 as a function of the slope value. In another suitable embodiment, data points close in time or any other suitable unit of measure may be ignored in calculating the slopes and in populating histogram 350. The dominant slope value, as shown by bar 360 in FIG. 5(b), may represent the dominant slope of the signal in plot 300, regardless of whether all of the data points in the signal were used to calculate the slope values. Due to their outlying nature, the slope values of the signal using data points D and G may be represented as a secondary slope on histogram 350, as shown by bar 375. It may be the case that bar 375 represents the desired slope and that the dominant slope given by bar 360 may be due to an erroneous (e.g., artifact) slope, which could happen if, for example, an artifact dominates the signal. Therefore, flexibility may be allowed to choose whichever local maxima in the histogram is desired. For example, a slope of 1 may be ignored, which may be present due to correlated noise. Also, as an example, the maxima corresponding to the highest slope may be selected under certain circumstances. Any suitable criteria may be used to select a slope according at least to any suitable information using any suitable techniques such as neural networks, empirically derived heuristics, weighted bin counts, any other suitable technique, or any combination thereof. Histogram 350 may allow all of the data points within plot 300 to be used in calculating the dominant slope of the signal, but may cause the outlying data points to appear as a non-dominant bar or bars. Histogram 350 therefore may be useful in not only determining the desired dominant slope of the signal in plot 300, but also in determining one or more secondary slopes contained within the signal due to any suitable secondary signal component or artifact (e.g., correlated noise). In an embodiment, histogram 350 may be smoothed using, for example, a smoothing technique such as Gaussian kernel smoothing, low pass filtering, or any other suitable means.

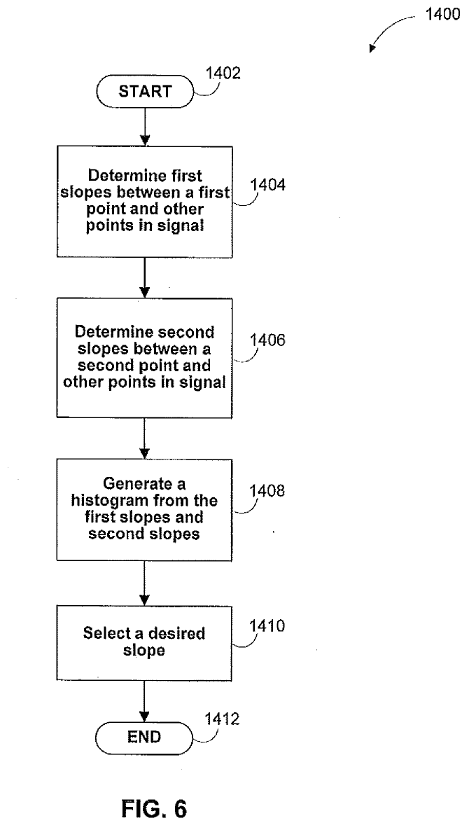

FIG. 6 is a flowchart of an illustrative process for determining a desired slope of a signal in accordance with an embodiment of the disclosure. Process 1400 may begin at step 1402. At step 1404, a first set of slope values is calculated between any first data point within the signal and other data points within the signal. Step 1404 may be performed by, for example, microprocessor 48 (FIG. 2) operating in real time on samples from QSM 72 (FIG. 2) or from samples stored in RAM 54 (FIG. 2). All data points may be included in determining the first set of slope values, which may include outlying data points. Alternatively, slope values may be calculated between any first data point and any other suitable number of data points (e.g., all data points not within a close proximity or other unit of measure of the first data point, all data points within a particular portion of the signal, or a fixed number of data points).

In an embodiment, process 1400 may advance to step 1406, in which a second set of slope values of the signal may be determined. The second set of slope values may be calculated in the same manner in which the first set of slope values was determined (e.g., using all of the data points within the signal), but using a second data point from which to determine the second set of slope values. In an embodiment, process 1400 may include any suitable number of additional steps (not shown) to determine any suitable number of sets of slope values for other data points in the signal. For example, the signal used in process 1400 may contain 100 data points, and process 1400 may include 100 steps to determine 100 sets of slope values before advancing to step 1408. In another suitable embodiment, process 1400 may include 4,950 steps to determine the slope between every combination of each of the 100 data points.

In an embodiment, process 1400 may advance to step 1408, in which the first set and the second set of slope values each may be used to generate a histogram. (e.g., by microprocessor 48 of FIG. 2). The histogram may include any suitable axes representing any suitable units of measure. In an embodiment, the histogram may plot the number of occurrences of each slope value as a function of the slope value. Each slope value may be represented in any suitable manner on the histogram, such as by a bar. The height of each bar on the histogram may correspond to the number of occurrences of slope that occur at the slope value or within the range of slope values for that bar value.

In an embodiment, process 1400 may advance to step 1410, in which a desired slope may be selected from the histogram. In an embodiment, it may be desirable to determine the dominant slope value of the signal. The slope value with the maximum number of occurrences on the histogram (e.g., the histogram bar with the maximum height) may indicate the signal's dominant slope value. In an embodiment, it may be desirable to determine another slope value within the signal. For example, the signal may contain other components or artifacts, such as correlated noise. The histogram may illustrate that one or more of these components has a particular slope value with a number of occurrences greater than other determined slope values, but not the greatest number of occurrences as compared with the dominant slope value of the signal. The histogram may allow this secondary slope value to be located and its value determined. In some embodiments, slope values may be automatically selected from the histogram by microprocessor 48 (FIG. 2) or processor 412 (FIG. 4) and then displayed on any suitable output mechanism, such as display 20 (FIG. 2), display 28, (FIG. 2) or output 414 (FIG. 4). In other embodiments, microprocessor 48 (FIG. 2) may display the histogram on display 20, display 28, or output 414, and the desired slopes may be selected or otherwise identified by a user via user inputs 56 (FIG. 2). Process 1400 may then advance to step 1412 and end. It is to be understood that process 1400 may be performed with any suitable signals, including signals that may form a Lissajous figure and may be obtained from one or more scalograms (described above with respect to FIGS. 3(a), 3(b), and 3(c)), and/or the original signals from which the scalograms may be derived.

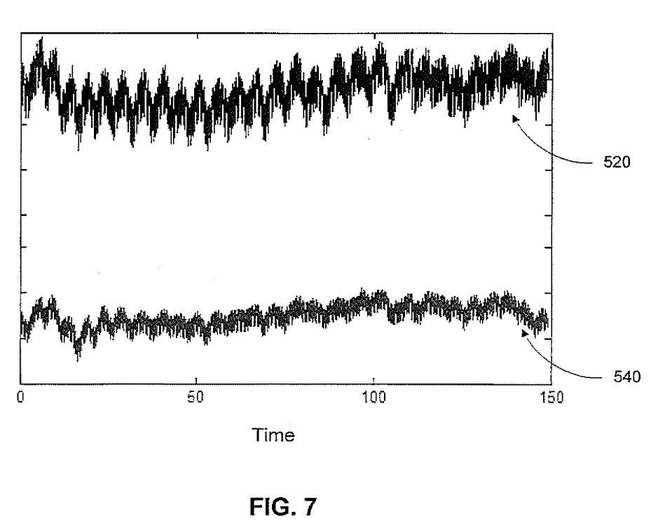

FIG. 7 shows a plot of two signals detected in accordance with an embodiment of the disclosure in which PPG signals are employed. In this embodiment, one PPG signal may include a red light signal 520 and another PPG signal may include an infrared light signal 540 obtained from a pulse oximeter sensor as described above. Red light signal 520 and infrared signal 540 may be plotted as shown in FIG. 7 after passing through a portion of the patient's blood perfused tissue (e.g., a fingertip, a toe, a foot). The pulse oximeter sensor may transmit red light signal 520 and infrared light signal 540 to any suitable processing unit (e.g., processor 412 in FIG. 4 or microprocessor 48 in FIG. 2) for further analysis. For example, analyzing the ratio between changes in the red light signal 520 and changes in the infrared light signal 540 after both signals have passed through human tissue may be useful in determining a patient's blood oxygen saturation.

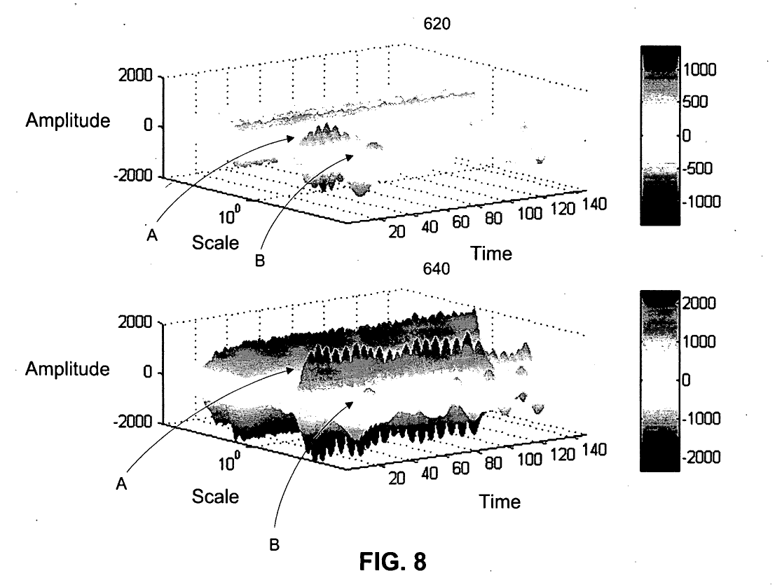

FIG. 8 shows the real part of the wavelet transform of each of the detected signals in FIG. 7 in accordance with an embodiment of the disclosure. The wavelet transform, from which FIG. 8 was derived, was computed using a complex wavelet. The imaginary parts of the wavelet transform, or the real part of the transform, and/or other forms of the transform, may also be utilized in the method. If, for example a wavelet with only a real component is used, then the transform itself may be used. The scalogram, as defined above, may also be utilized. However, the real-only (or imaginary-only) parts of the transform may produce more pronounced oscillatory behavior of the transform surface, which enhances the technique. All such transforms that may be used are herein referred to as a “transform-surface.” A transform-surface may or may not be continuous. In an embodiment, a transform-surface may be represented in a three-dimensional array. Transform-surface 620 and transform-surface 640 may be derived using any suitable method. In an embodiment, each of transform-surface 620 and transform-surface 640 may be the result of taking a transform modulus of red light signal 520 and infrared light signal 540, respectively, as described above with respect to FIGS. 3(a), 3(b), and 3(c).

In an embodiment, transform-surface 620 and transform-surface 640 may each include any suitable number of ridges at any suitable scale value. In an embodiment, red light signal 520 and infrared light signal 540 may each include components relating to the pulse of a patient, the breathing rate of a patient, and one or more signal artifacts (e.g., noise). The signal components related to pulse rate and breathing rate may contain higher energy than other signal components. Transform-surface 620 and transform-surface 640 may each include a ridge at a particular scale value that may be related to the pulse component and the breathing component of red light signal 520 and infrared light signal 540, respectively. In each of transform-surface 620 and transform-surface 640, arrow A may indicate a ridge related to the pulse component of each signal, and arrow B may indicate a ridge related to the breathing component of each signal. Other ridges shown in transform-surfaces 620 and 640 may indicate other components of signals 520 and 540, such as high- or low-frequency noise.

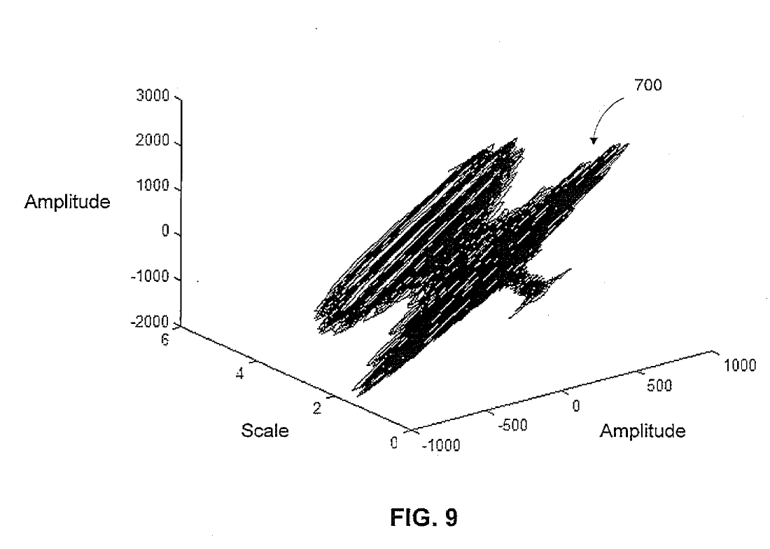

FIG. 9 shows a three-dimensional Lissajous figure derived at least in part from the transform-surfaces of FIG. 8 in accordance with an embodiment of the disclosure. In an embodiment, the surfaces of transform-surfaces 620 and 640 may be plotted against each other over a range of scales using, for example, a sliding time window (not shown). For each scale value, the amplitude surface of transform-surface 620 may be plotted against the amplitude surface of transform-surface 640 to derive a Lissajous figure. For example, one Lissajous figure may result from plotting the changes in amplitude of transform-surface 620 against the changes in amplitude of transform-surface 640 for each particular scale value over a period of time. A three-dimensional Lissajous FIG. 700 may be assembled from plotting all of the Lissajous figures of transform-surfaces 620 and 640 for a given time period.

In an embodiment, FIG. 700 may be analyzed further to select one Lissajous figure using any suitable method. For example, one Lissajous figure within FIG. 700 may have the greatest length along its major axis and the least spread perpendicular to the major axis. The dimensions of this selected Lissajous figure may indicate a low amount of noise or a high signal to noise ratio in signals 520 and 540 at the scale corresponding to the selected Lissajous figure.

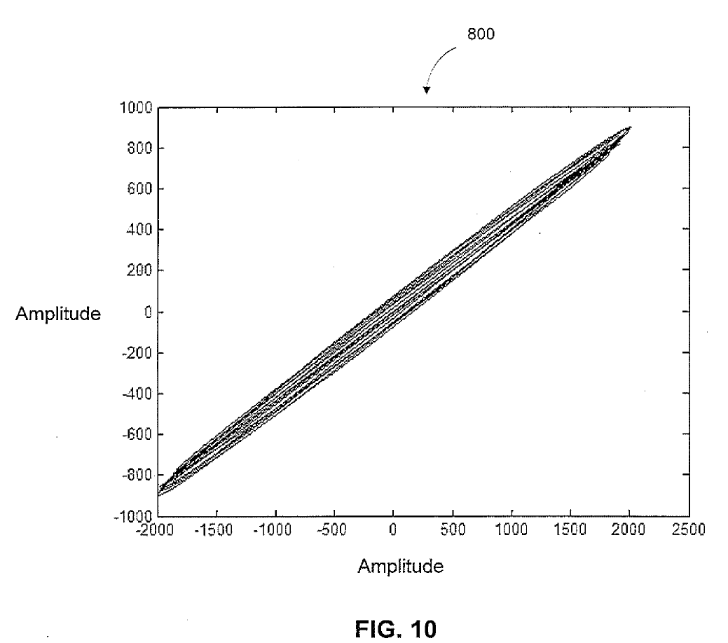

FIG. 10 shows a Lissajous figure selected from the three-dimensional Lissajous figure of FIG. 9 in accordance with an embodiment of the disclosure. Lissajous FIG. 800 may be selected from three-dimensional Lissajous FIG. 700 because it may include the greatest length along its major axis and the least spread perpendicular to the major axis, thereby indicating a minimal amount of associated noise. In FIG. 10, the amplitude of transform-surface 620 may be plotted as a function of the amplitude of transform-surface 640 for the scale corresponding to the selected Lissajous FIG. 800 over a period of time. In an embodiment, Lissajous FIG. 800 may have a dominant slope that in an embodiment may have relevance. For example, if three-dimensional Lissajous FIG. 700 was created using transform-surfaces 620 and 640 based at least in part on red light signal 520 and infrared light signal 540, respectively, then the dominant slope of FIG. 800 may correspond to a ratio between changes in red light signal 520 and changes infrared light signal 540. The ratio may be used with a look-up table of values or used in a calibration equation to determine the blood oxygen saturation level of a patient from which red light signal 520 and infrared light signal 540 were obtained. The lookup table may be created, for example, using empirical patient data. It is to be understood from this disclosure that using Lissajous FIG. 800 to determine a patients blood oxygen saturation level does not require a determination of the patient's pulse rate. In an embodiment, however, the scale at which the patient's pulse rate may manifest itself in transform-surface 620 and/or transform-surface 640 may be used to select FIG. 800 from three-dimensional Lissajous FIG. 700.

In an embodiment, to determine the dominant slope of FIG. 800, a least squares technique may be applied to create a best-fit line using the data points within FIG. 800. The least squares technique may be skewed, however, by the presence of other signal components, such as correlated noise that may be present within signals 520 and 540. The presence of the other signal components may represent a secondary slope within FIG. 800 that may skew the determination of the dominant slope and may affect the analysis of patient information using the dominant slope.

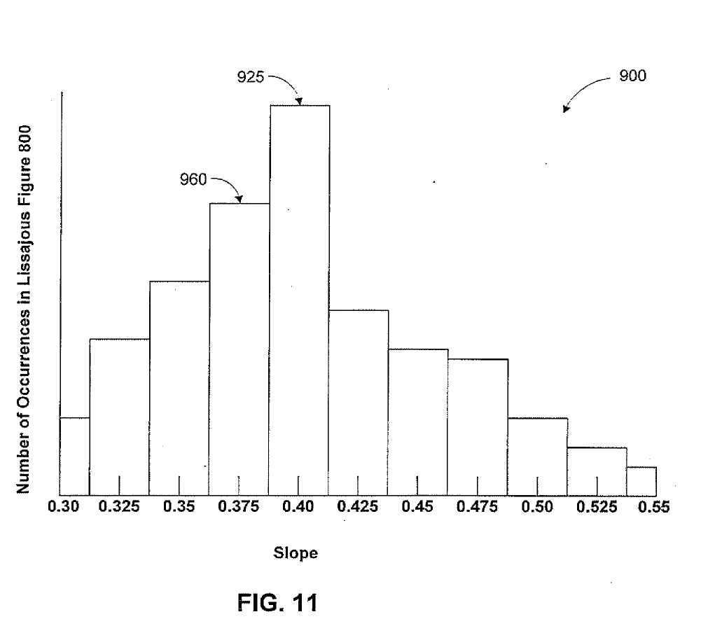

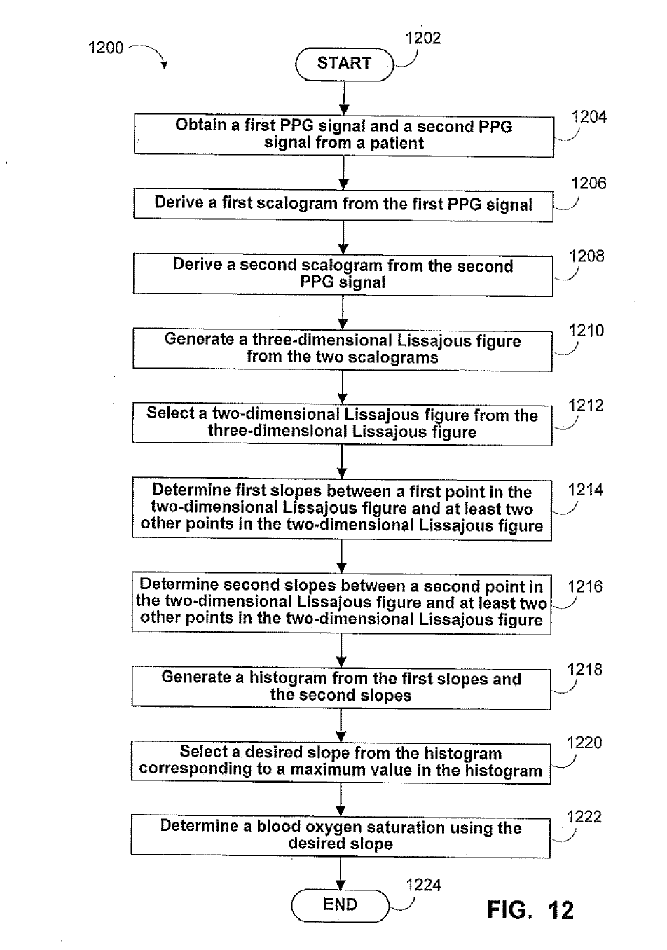

In an embodiment, the dominant slope, which may be related to the dominant components (e.g., the components with the highest energy) within signals 520 and 540, may be determined using any suitable approach. FIG. 11 shows a histogram of the slopes or range of slope values of FIG. 800 in accordance with an embodiment of the disclosure. In an embodiment, the slope of FIG. 800 between each of the data points of FIG. 800, including any outlying data points, may be determined. In an embodiment, data points close in time or any other suitable unit of measure may be ignored in calculating the slopes of FIG. 800. The resulting calculations may be graphed in any suitable manner, such as by creating histogram 900. Histogram 900 may represent graphically the number of times that a particular slope value or range of slope values may occur within FIG. 800. The maximum of histogram 900, shown at dominant slope bar 925, may indicate the dominant slope of FIG. 800. In an embodiment, dominant slope 925 may be used with a look-up table or used in a calibration equation to determine a patient's blood oxygen saturation level. In an embodiment, histogram 900 may be smoothed using, for example, a smoothing technique such as Gaussian kernel smoothing, low pass filtering, or any other suitable means.

In an embodiment, histogram 900 may also represent graphically one or more other slope values or range of slope values present within FIG. 800 due to one or more other components within signal 520 and/or signal 540. By using histogram 900 to ascertain the dominant slope of FIG. 800, the one or more secondary slopes or range of secondary slope values represented, for example by histogram bar 960, may not skew the determination of the dominant slope of FIG. 800. In an embodiment, the slopes between data points close in time or any other suitable unit of measure may not be plotted on histogram 900 to remove some of the effect of the other signal components. Alternatively, the presence or relative impact of one or more secondary slopes may be of interest. For example, a secondary slope within FIG. 900 (e.g., histogram bar 960) that may be related to noise within signals 520 and 540 may be analyzed by creating histogram 900.

FIG. 12 is a flowchart of an illustrative process for determining a blood oxygen saturation of a patient in accordance with an embodiment. Process 1200 may begin at step 1202. At step 1204, a first PPG signal and a second PPG signal may be obtained from a patient using any suitable method. For example, the first and second PPG signals may be obtained from sensor 12 that may be coupled to patient 40 (FIG. 2). Alternatively, the PPG signals may be obtained from input signal generator 410, which may include oximeter 420 coupled to sensor 418, which may provide as input signal 416 FIG. 4) PPG signals. In an embodiment, the PPG signals may be obtained from patient 40 using sensor 12 or input signal generator 410 in real time. In an embodiment, the PPG signals may have been stored in ROM 52, RAM 52, and/or QSM 72 (FIG. 2) in the past and may be accessed by microprocessor 48 within monitor 14 to be processed. In an embodiment, the first PPG signal may include a red light signal and the second PPG signal may include an infrared light signal. The first and second PPG signals may he obtained simultaneously from patient 40.

In an embodiment, process 1200 may advance to step 1206, where a first scalogram may be derived at least in part from the first PPG signal Process 1200 may advance to step 1208, where a second scalogram may be derived at least in part from the second PPG signal. In an embodiment step 1208 may occur simultaneously with step 1206. For example, the scalograms may be derived as described above with respect to FIGS. 3(a), 3(b), and 3(c) after the PPG signals have been sent to microprocessor 48 or alternatively, to processor 412 (FIG. 4). In an embodiment, processor 412 or microprocessor 48 may perform the calculations associated with the continuous wavelet transforms of the PPG signals.

In an embodiment, process 1200 may advance to step 1210, where a three-dimensional Lissajous figure may be generated from the two scalograms. For example, the three-dimensional Lissajous figure may be generated as described above with respect to FIG. 9. One Lissajous figure may result from plotting the changes in amplitude of, for example, the first scalogram against the changes in amplitude of the second scalogram for each particular scale value over a period of time. A three-dimensional Lissajous figure may be assembled from plotting all of the Lissajous figures of the scalograms for a given time period. In an embodiment, processor 412 or microprocessor 48 may include any suitable software, firmware, and/or hardware, and/or combinations thereof for processing the scalograms of steps 1206 and 1208 to generate the three-dimensional Lissajous figure.

In an embodiment, process 1200 may advance to step 1212, where a two-dimensional Lissajous figure may be selected from the three-dimensional Lissajous figure as described above with respect to FIG. 10. The two-dimensional Lissajous figure may be selected from the three-dimensional Lissajous figure because it may include the greatest length along its major axis and the least spread perpendicular to the major axis, thereby indicating a minimal amount of associated noise. The two-dimensional Lissajous figure may have a dominant slope that has clinical relevance (e.g., the slope may be used with a look-up table of values or in a calibration equation to determine the blood oxygen saturation level of a patient from which the PPG signals were obtained at step 1204) because the dominant slope of the two-dimensional Lissajous figure may correspond to a ratio between changes in the red light signal and infrared light signal. In an embodiment, processor 412 or microprocessor 48 may include any suitable software, firmware, and/or hardware, and/or combinations thereof for isolating the desired two-dimensional Lissajous figure from the three-dimensional Lissajous figure.

In an embodiment, process 1200 may advance to step 1214, where a first set of slopes may be determined between a first point of the two-dimensional Lissajous figure and at least two other data points in the Lissajous figure using any suitable approach. In an embodiment, each data point may be used at step 1214. In an embodiment, data points close in time, proximity, or any other suitable unit of measure to one another may not be used and outlying data points may also not be used. At step 1216, a second set of slopes may be calculated between a second point of the two-dimensional Lissajous figure and at least two other data points in the Lissajous figure using any suitable approach. As with step 1214, each data point may be used at step 1216 or alternatively, data points close in time, proximity, or any other suitable unit of measure to one another may not be used and outlying data points may also not be used. In an embodiment, processor 412 or microprocessor 48 may include any suitable software, firmware, and/or hardware, and/or combinations thereof for determining the slope values and determining which data points to use in calculating the slope values. In an embodiment, process 1200 may include any suitable number of additional steps (not shown) to determine any suitable additional number of sets of slope values for other data points in the signal.

In an embodiment, process 1200 may advance to step 1218, where a histogram may be generated from the first set of slopes calculated in step 1214 and the second set of slopes calculated in step 1216 using any suitable approach. In an embodiment, processor 412 or microprocessor 48 may include any suitable software, firmware, and/or hardware, and/or combinations thereof for generating the histogram at step 1218 from the first and second sets of slope values. The histogram may represent graphically the number of times that a particular slope value or range of slope values may occur within the two-dimensional Lissajous figure. The maximum of the histogram may indicate the dominant slope of the two-dimensional Lissajous figure. The histogram may be displayed in any suitable manner, such as on display 20 (FIG. 2), display 28 (FIG. 2) or using output 414, for review by patient 40, a user of system 10 or system 400, and/or a medical professional treating patient 40.

In an embodiment, process 1200 may advance to step 1220, where a desired slope may be selected from the histogram using any suitable approach and the desired slope may correspond to the maximum value in the histogram. The maximum value of the histogram may be displayed in any suitable manner, such as on display 20, display 28, or using output 414. In an embodiment, processor 412 or microprocessor 48 may include any suitable software, firmware, and/or hardware, and/or combinations thereof for automatically identifying and displaying the maximum value of the histogram. In an embodiment, patient 40, a user of system 10 or system 400, and/or a medical professional treating patient 40 may review the displayed histogram and may identify the maximum value of the histogram, and thereby identify the desired slope, visually. In an embodiment, output 414 or pulse oximetry system 10 may present the maximum value of the histogram as an audio output to speaker 22 (FIG. 2) (e.g., a spoken statement).

In an embodiment, process 1200 may advance to step 1222, where the desired slope may be used with a look-up table or in a calibration equation to determine a patient's blood oxygen saturation level. In an embodiment, processor 412 or microprocessor 48 may include any suitable software, firmware, and/or hardware, and/or combinations thereof for determining the blood oxygen saturation of the patient 40. For example, processor 412 or microprocessor 48 may incorporate a look-up table or the calibration equation and may determine the blood oxygen saturation using the desired slope value identified from the histogram in step 1220. In an embodiment, the maximum value of the histogram identified in step 1220 may not be displayed on display 20, display 28, or output 414, as the maximum value may not have clinical relevance apart from the look-up table or the calibration equation. In an embodiment, a user of pulse oximetry system 10 or continuous wavelet processing system 400 may use the desired slope displayed at step 1220 in conjunction with a look-up table or in a calibration equation to determine the blood oxygen saturation of patient 40. The blood oxygen saturation of patient 40 may be displayed in any suitable manner, such as on display 20, display 28, or using output 414. In an embodiment, pulse oximetry system 10 or continuous wavelet processing system 400 may present the blood oxygen saturation as an audio output to speaker 22 (e.g., a spoken statement). Process 1200 may then,advance to step 1224 and end.

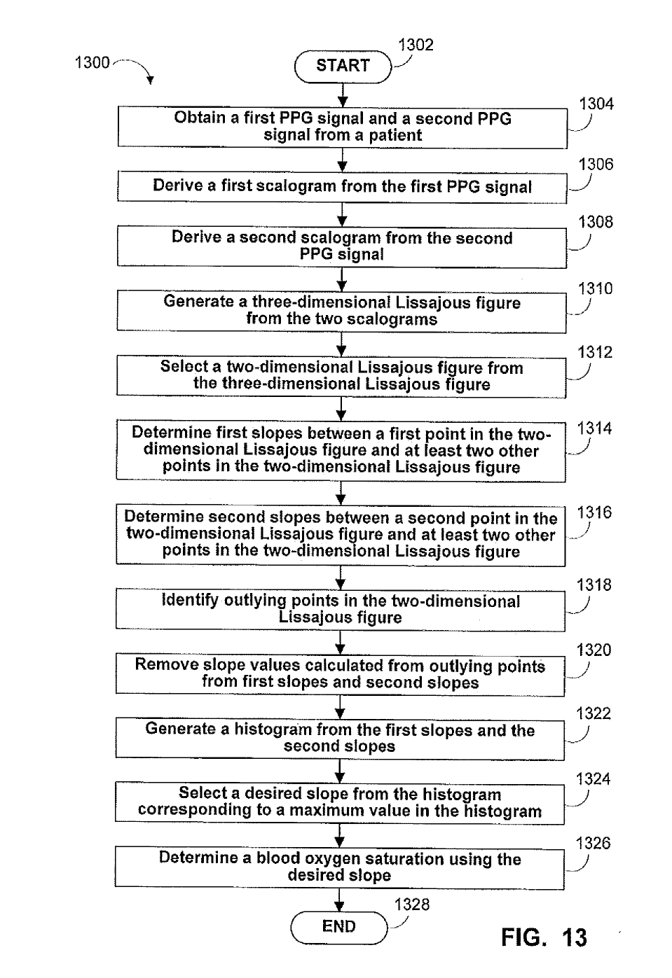

In an embodiment, Lissajous FIG. 800 may be used in conjunction with one or more algorithms intended to detect or reduce noise using any suitable approach. FIG. 13 is a flowchart of an illustrative process for determining a blood oxygen saturation of a patient after a noise algorithm is applied to a two-dimensional Lissajous figure in accordance with an embodiment. Process 1300 may begin at step 1302. Steps 1304 through 1316 may be the same as, and may be performed by the same systems or using the same methods as, steps 1204 through 1216 as described above with respect to FIG. 12.

In an embodiment, at step 1318, at least one point in the two-dimensional Lissajous figure may be identified. This at least one point may be an offshoot, or an outlying point, of the two-dimensional Lissajous figure. The offshoot may result from any suitable cause, such as noise generated by input signal generator 410 (FIG. 4) or noise generated by system 10, including for example, sensor 12, monitor 14, display 28, or cables 32 and/or 24 (FIG. 2). The outlying point or points may be identified at any suitable time, regardless of when the first and second PPG signals may have been obtained from patient 40. For example, noise may be detected in real time as the PPG signals are obtained from patient 40 by examining the two-dimensional Lissajous figure for outlying data points at step 1318. In an embodiment, the noise may be detected in real time using a separate noise determination component. Alternatively, the first and second PPG signals may have been stored in ROM 52, RAM 52, and/or QSM 72 (FIG. 2) in the past and may be accessed by microprocessor 48 to be analyzed using process 1300. Outlying data points may be identified in step 1318 in any suitable manner. In an embodiment, outlying data points may be identified by comparing the slope values calculated in steps 1314 and/or 1316 using adjacent or approximately adjacent data points. For example, slope values that are significantly different than the majority of adjacent or approximately adjacent slope values in the first set of slopes calculated in step 1314 or in the second set of slopes calculated in step 1316 may indicate the presence of one or more outlying data points. In an embodiment, processor 412 or microprocessor 48 may include any suitable software, firmware, and/or hardware, and/or combinations thereof for comparing slope values to locate at least one outlying point in the two-dimensional Lissajous figure.

In an embodiment, process 1300 may advance to step 1320, where the slope values calculated in steps 1314 and/or 1316 from any of the points identified in step 1318 as outlying data points of the two-dimensional Lissajous figure may be removed from the first and second sets of slope values. In an embodiment, processor 412 or microprocessor 48 may include any suitable software, firmware, and/or hardware, and/or combinations thereof for manipulating the first and second sets of slope values to remove any slope values that may include one or more outlying points identified in step 1318.

In an embodiment, process 1300 may advance to step 1322, where a histogram may be generated from the first set of slopes calculated in step 1314 and the second set of slopes calculated in step 1316, but excluding the slope values calculated using any of the identified outlying data points, using any suitable approach. In an embodiment, processor 412 or microprocessor 48 may include any suitable software, firmware, and/or hardware, and/or combinations thereof for generating the histogram at step 1318 from the first and second sets of slope values. The histogram may be displayed in any suitable manner, such as on display 20 (FIG. 2), display 28 (FIG. 2) or using output 414 (FIG. 4), for review by patient 40, a user of system 10 or system 400, and/or a medical professional treating patient 40. The histogram may represent graphically the number of times that a particular slope value or range of slope values may occur within the two-dimensional Lissajous figure. The maximum of the histogram may indicate the dominant slope of the two-dimensional Lissajous figure. Because the slope values calculated using the outlying points or the points may not be plotted on the histogram, a secondary slope may not appear on the histogram related to noise, thereby further reducing the effect of noise on the calculation of the desired slope.

In an embodiment, process 1300 may advance to steps 1324 and 1326, which may be the same as, and may be performed by the same systems or using the same methods as, steps 1220 and 1222 as described above with respect to FIG. 12. Process 1300 may then advance to step 1328 and end.

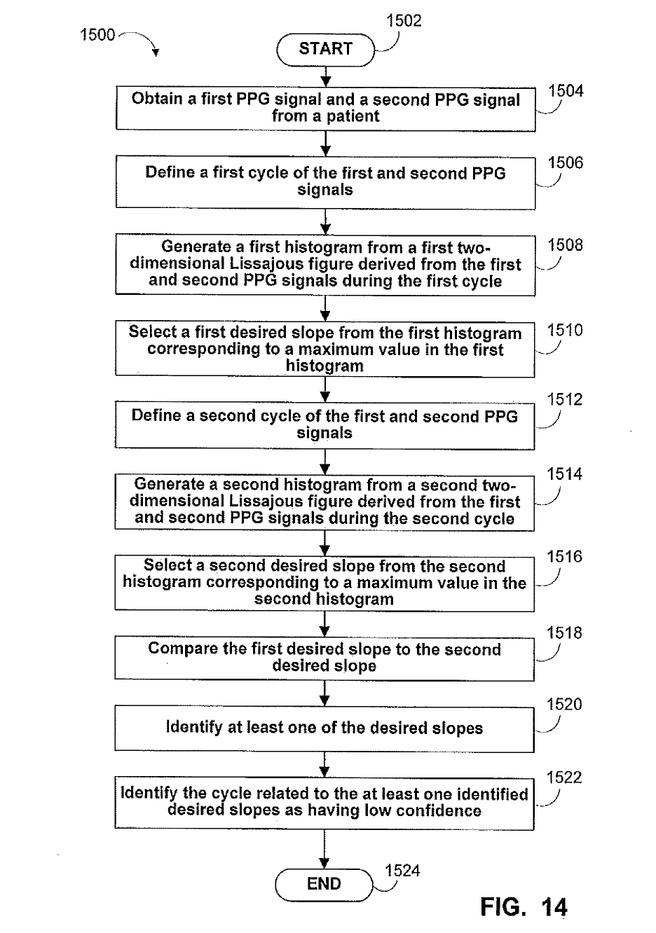

In an embodiment, a confidence measure may be generated in conjunction with determining dominant slope 925. FIG. 14 is a flowchart of an illustrative process for generating a confidence measure in a histogram in accordance with an embodiment. Process 1500 may begin at step 1502. At step 1504, a first PPG signal and a second PPG signal may be obtained from a patient using any suitable method. For example, the first and second PPG signals may be obtained from sensor 12 that may be coupled to patient 40 (FIG. 2). Alternatively, the PPG signals may be obtained from input signal generator 410, which may include oximeter 420 coupled to sensor 418, which may provide as input signal 416 (FIG. 4) PPG signals. In an embodiment, the PPG signals may be obtained from patient 40 using sensor 12 or input signal generator 410 in real time. In an embodiment, the PPG signals may have been stored in ROM 52, RAM 52, and/or QSM 72 (FIG. 2) in the past and may be accessed by microprocessor 48 within monitor 14 to be processed. In an embodiment, the first PPG signal may include a red light signal and the second PPG signal may include an infrared light signal. The first and second PPG signals may be obtained simultaneously from patient 40.

In an embodiment, process 1500 may advance to step 1506, in which a first cycle may be defined for the first and second PPG signals. For example, a cycle may be defined by any suitable mechanism (e.g., by microprocessor 48 or by processor 412) as a finite period of time over which transform-surface 620 and transform-surface 640 (FIG. 8) may be created. As a result, at step 1508, a three-dimensional Lissajous figure, a first two-dimensional Lissajous figure, and a first histogram each may be derived at least in part as described above with respect to steps 1206 through 1218 of FIG. 12 from the portion of the first and second PPG signals collected during the first cycle. Process 1500 may advance to step 1510, where a first desired slope may be selected from the first histogram, and the first desired slope may correspond to a maximum value in the first histogram. In an embodiment, the first desired slope may have clinical relevance, as it may be input into a look-up table or used in a calibration equation to determine a blood oxygen saturation level of a patient 40 from whom the PPG signals were obtained. In an embodiment, processor 412 or microprocessor 48 may include any suitable software, firmware, and/or hardware, and/or combinations thereof for automatically selecting the maximum value of the first histogram.

In an embodiment, process 1500 may advance to step 1512, where at the end of the first cycle, a second cycle may be defined. In an embodiment, the second cycle may overlap the first cycle or be spaced apart from the first cycle. Process 1500 may advance to step 1514, where a second three-dimensional Lissajous figure, a second two-dimensional Lissajous figure, and a second histogram may be derived at least in part as described above with respect to steps 1206 through 1218 of FIG. 12 from the portion of the first and second PPG signals collected during the second cycle. At step 1516, a second desired slope may be selected from the second histogram, and the second desired slope may correspond to a maximum value in the second histogram. As with the first desired slope, the second desired slope also may have clinical relevance related to a patients blood oxygen saturation level. In an embodiment, processor 412 or microprocessor 48 may include any suitable software, firmware, and/or hardware, and/or combinations thereof for automatically selecting the maximum value of the second histogram. This process 1500 of deriving successive Lissajous figures and related histograms from successive transform-surfaces may continue for any additional desired number of cycles (not shown).

In an embodiment, process 1500 may advance to step 1518, where the first desired slope and the second desired slope obtained from the first and second cycles may be compared. In an embodiment, processor 412 or microprocessor 48 may include any suitable software, firmware, and/or hardware, and/or combinations thereof for making a comparison between any suitable number of slope values. At step 1520, at least one of the desired slopes may be identified. In an embodiment, one cycle may include a histogram that may generate a significantly different desired slope value, if for example the data from the PPG signals used to generate the histogram may have been distorted by patient movement, a poor connection between patient 40 and sensor 12, or was otherwise faulty. It would be undesirable to use the significantly different desired slope value to calculate the blood oxygen saturation level of patient 40. Therefore, it would be beneficial to identify the one or more portions of the PPG signals that contain the data points leading to the significantly different desired slope value and to remove those portions from further analysis of the PPG signals in relation to determining blood oxygen saturation. The desired slope value that may be significantly different than the one or more other desired slope values to which it is being compared may be automatically identified by microprocessor 48 or processor 412 using any suitable technique.

In an embodiment, process 1500 may advance to step 1522, where the cycle that may be related to the identified desired slope value may be identified as having a low confidence as compared to the other cycles and resulting histograms that may have been generated from the first and second PPG signals. In an embodiment, the data from the PPG signals that may be included in the cycle identified as having low confidence may be removed from further analysis by a user of system 10 or system 400 or automatically by microprocessor 48 or processor 412. Process 1500 may then advance to step 1524 and end.

In an embodiment, the shape of histogram 900 may be used to measure confidence in the calculation of dominant slope 925. For example, dimensions such as the height of dominant slope 925 on histogram 900, or the width of histogram 900, may be used to measure a confidence level as to whether the value of dominant slope 925 is reliable. Alternatively, the ratio of the area underneath dominant value 925 to the area underneath histogram 900 using the entire data set of histogram 900 may also be used to determine whether the calculation of dominant value 925 is reliable.

In an embodiment, the value of the dominant slope from a histogram may be used for line fitting with respect to the original signal using any suitable method. For example, in a line fitting equation such as y=a+bx, dominant slope 925 may represent the “b” value. The offset of the line, represented by the “a” value, may be calculated using any suitable method. In an embodiment, the offset may be calculated by examining the data points that were used to calculate the slope values that fall within the dominant slope represented on the histogram. The data points that were not used to calculate the slope values that fall within the dominant slope may be ignored in calculating the offset value. This may exclude one or more outlying data points from affecting the calculation of the offset for the best-fit line.

The foregoing is merely illustrative of the principles of this disclosure and various modifications can be made by those skilled in the art without departing from the scope and spirit of the disclosure.

What is claimed is:

1. A method for determining a desired slope of a signal, comprising:
determining first slopes between a first point in the signal and at least two other points in the signal;
determining second slopes between a second point in the signal and at least two other points in the signal;
generating a histogram from the first slopes and the second slopes; and
selecting the desired slope of the signal corresponding to a preferred value in the histogram.

2. The method of claim 1, further comprising:
selecting at least a portion of the signal;
determining the first slopes between the first point and every combination of other points within the selected portion of the signal; and
determining the second slopes between the second point and every combination of other points within the selected portion of the signal.

3. The method of claim 1, further comprising excluding at least one point in the signal from the determined first slopes and the determined second slopes.

4. The method of claim 1, wherein the signal is a Lissajous figure.

5. The method of claim 4, further comprising selecting the Lissajous figure from a three-dimensional Lissajous figure.

6. The method of claim 5, further comprising:
performing a first continuous wavelet transform on a first underlying signal to derive a first scalogram;
performing a second continuous wavelet transform on a second underlying signal to derive a second scalogram; and
deriving the three-dimensional Lissajous figure from the first scalogram and the second scalogram.

7. The method of claim 6, wherein the first underlying signal is a red light signal collected by a pulse oximeter from a patient, and wherein the second underlying signal is an infrared light signal collected by the pulse oximeter from the patient.

8. The method of claim 7, further comprising determining blood oxygen saturation information about a patient based at least in part on the desired slope.

9. The method of claim 1, further comprising generating a confidence measure from the histogram.

10. The method of claim 1, further comprising filtering the signal with a noise reduction algorithm.

11. A system for determining a desired slope of a signal, the system comprising:
an input signal generator for generating a signal;
a processor coupled to the input signal generator; and
an output coupled to the processor, wherein the output is capable of displaying information based at least in part on the desired slope and wherein the processor is capable of:
determining first slopes between a first point in the signal and at least two other points in the signal;
determining second slopes between a second point in the signal and at least two other points in the signal;
generating a histogram from the first slopes and the second slopes; and
selecting the desired slope of the signal corresponding to a preferred value in the histogram.

12. The system of claim 11, wherein the processor is further capable of:
selecting at least a portion of the signal;
determining the first slopes between the first point and every combination of other points within the selected portion of the signal; and
determining the second slopes between the second point and every combination of other points within the selected portion of the signal.

13. The system of claim 11, wherein the processor is further capable of excluding at least one point in the signal from the determined first slopes and the determined second slopes.

14. The system of claim 11, wherein the input signal generator is a pulse oximeter coupled to a sensor.

15. The system of claim 14, wherein the processor is further capable of determining blood oxygen saturation information about a patient based at least in part on the desired slope.

16. The system of claim 11, wherein the output is an electronic device.

17. The system of claim 11, wherein the processor is further capable of generating a confidence measure using the histogram.

18. The system of claim 11, wherein the processor is further capable of filtering the signal by performing a noise reduction algorithm.

19. A computer-readable medium for use in determining a desired slope of a signal, the computer-readable medium having computer program instructions recorded thereon for:
determining first slopes between a first point in the signal and at least two other points in the signal;
determining second slopes between a second point in the signal and at least two other points in the signal;
generating a histogram from the first slopes and the second slopes; and
selecting the desired slope of the signal corresponding to a preferred value in the histogram.

20. The computer-readable medium of claim 19, further having computer program instructions recorded thereon for obtaining the signal using a pulse oximeter.

21. The computer-readable medium of claim 20, further having computer program instructions recorded thereon for:
determining blood oxygen saturation information based at least in part on the desired slope; and
displaying a blood oxygen saturation level based using the desired slope.

22. The computer-readable medium of claim 21, wherein the determining blood oxygen saturation information comprises using the desired slope in a look-up table.

23. The computer-readable medium of claim 21, wherein the determining blood oxygen saturation information comprises using the desired slope in a calibration equation.
